(19) United States
(12) Patent Application Publication
(10) Pub. No.: US 2025/0064397 A1
(43) Pub. Date: Feb. 27, 2025

(54) METHOD AND WEARABLE TRACKER SYSTEM FOR HEALTH CONDITIONS AND DIAGNOSIS

(71) Applicant: ROSALIND FRANKLIN UNIVERSITY OF MEDICINE AND SCIENCE, North Chicago, IL (US)

(72) Inventor: Holly HUNSBERGER, Arlington Heights, IL (US)

(21) Appl. No.: 18/944,999

(22) Filed: Nov. 12, 2024

Related U.S. Application Data
(63) Continuation of application No. PCT/US2023/069695, filed on Jul. 6, 2023.
(60) Provisional application No. 63/359,188, filed on Jul. 7, 2022.

(51) Int. Cl. A61B 5/00; A61B 5/0205

(52) U.S. Cl. CPC A61B 5/6822; A61B 5/002; A61B 5/0205; A61B 5/6823; A61B 5/7246; A61B 5/4064; A61B 5/4088; A61B 5/4094; A61B 5/4842; A61B 2503/40; A61B 2560/0209

(57) ABSTRACT

Provided herein are systems, methods and apparatuses for a miniaturized electronic device worn around the subject tracks health data for diagnosis and preclinical studies.

### CROSS-REFERENCE TO RELATED APPLICATIONS

The present application claims priority to PCT application serial no. PCT/US2023/069695, filed on Jul. 6, 2023, which claims priority to U.S. provisional application Ser. No. 63/359,188, filed on Jul. 7, 2022, each herein incorporated by references in their entireties.

### BACKGROUND

The present invention relates generally to a miniaturized electronic device that tracks health data across a lifespan of a subject.

Mice and rats have long served as the preferred species for biomedical research, making up 99.3% of all mammals used. Well over 21 million mice & rats are used in the US annually to perform foundational scientific research and validate products across almost every disease state, drug, and medical intervention. Data also indicate that mice make up over 97% of these rodents used (1). Specific to epilepsy, mice studies represent the basis for foundational studies and represent strong candidate organisms for modeling human neurogenetic disorders with over 99% of mouse genes having human homologues (2). To use mice in epilepsy research, researchers rely on two methods to express epilepsy, genetic mice and induced epilepsy. Genetically predisposed animals more similarly mimic epilepsy found in people, but have greater variability in the onset of symptoms. “Induced models can be relied upon to exhibit acute or chronic seizures ‘on demand’ and therefore have high face validity, but their relevance to human epilepsies remains contentious (3).”

There are currently technologies in rodents including, wireless optogenetic brain implants, wearable blood flow monitor, machine learning life expectancy, a wearable pet scanner, and sleep boxes.

Current technologies to monitor epilepsy include electroencephalogram (EEG), behavior, wireless or wired telemetry; all having significant limitations which favor the use of induced epilepsy. In most mouse models of disease, there is an outward manifestation of the disorder which can be measured easily, but trying to model a disorder with a phenotype that only appears sporadically and briefly as in epilepsy proves challenging and could benefit from technology that could detect seizures dynamically in real time without the experimenter present or actively recording (3).

While Alzheimer's disease (AD) was discovered 110 years ago, there are currently only 4 approved drugs for symptomatic treatment. As of July 2020, the NIA supports 233 active clinical trials; however, a majority of clinical trials of have failed to meet endpoints. There are several reasons for the lack of translatability between preclinical and clinical drug studies, but a major limitation is the difficulty for academic labs to run high throughput intervention experiments. There is an overreliance on behavioral tests that are brief and sensitive to external factors. Additionally, to measure physiological changes or neuronal changes, researchers are forced to sacrifice the animals at different time points, creating cross sectional snapshots of the disease or intervention. Therefore, there is a need for longitudinal rodent studies to track the effectiveness of treatments as well as natural aging.

Post-traumatic epilepsy (PTE) accounts for 4-9% of all epilepsy cases and can range up to 50% among war veterans (7-10). Clinicians currently use regression methods based on TBI severity and other factors to create a risk ratio but these methods are inconvenient. While there is currently no device to predict seizure onset, others have attempted to use artificial neural networks (ANN) to predict PTE. The ANN model can predict the risk of PTE and is superior to the risk estimated based on traditional statistical methods. However, the calibration of the model is poor and requires large human sample sizes for data input (11).

However, these devices offer little to no physiological data, are very expensive, and are slightly invasive by injecting a chip to track the animal throughout its environment.

The present invention attempts to solve these problems, as well as others.

### SUMMARY OF THE INVENTION

Provided herein are systems, methods and apparatuses for a miniaturized electronic device worn around the subject, which incorporates a microprocessor and internet connection to track health data.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53 (c) EPC and Rule 28 (b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### BRIEF DESCRIPTION OF THE DRAWINGS

In the accompanying figures, like elements are identified by like reference numerals among the several preferred embodiments of the present invention.

FIGS. 1A-1D are photographs of the wearable tracker on rats in home cage and behavior tests.

FIG. 2 is a photograph showing a phone used to collect continuous data from the wearable tracker.

FIGS. 3A-3B are graphs showing the wearable tracker detecting temperature across 24 hrs;

FIG. 3A is a graph showing the average temperature readings while in a home cage during light phase (sleep) and dark phases (wake); and FIG. 3B is a graph showing the continuous temperature reading throughout the day.

FIGS. 4A-4B are graphs showing the wearable tracker allowing typical mobility in an open field test; FIG. 4A are representative heat maps of movement comparing rats wearing sensors vs. a control rat without a sensor; and FIG. 4B are distance, traveled, time mobile, and temperature were similar among the groups.

FIGS. 5A-5B are graphs showing the wearable tracker sensors allow typical mobility in an elevated plus maze test; FIG. 5A are representative heat maps of movement comparing rats wearing sensors vs. a control rat without a sensor; and FIG. 5B are graphs showing distance, open arm time, and temperature were similar among the groups.

FIG. 6 is a graph showing the wearable tracker detecting temperature across multiple days.

FIG. 7 is a Wearable Health Tracker in wireless communication with a computer or smart phone.

FIG. 8 is a schematic showing the wearable tracker components.

FIG. 9A-9D are graphs showing the alterations in synaptic transmission post-TBI (7-10d) in male rodent hippocampus. FIG. 9A is a graph showing the input-out curve, a measure of synaptic strength, are increased post TBI (single and repeat models) indicating dysregulation of synaptic circuits, and affects memory network fidelity. FIG. 9B is a graph showing the PTP, a short term form of pre-synaptic plasticity, is also upregulated vs. sham controls, suggesting gating is imbalanced. FIG. 9C is a graph showing the LTP however is blunted in TBI (black circles). FIG. 9D is a graph showing consistent with FIG. 9B there are increased presynaptic fiber volleys in the TBI animals, suggesting hyperexcitability in the CA3 Schaffer collateral axons.

FIG. 10 is a photograph of mouse brain tissue damage post repeat TBI (white arrow).

FIG. 11 is a schematic flow diagram of the experimental behavioral design for Epilepsy.

FIG. 12 is a schematic flow diagram of the experimental design for Alzheimer's disease.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the biological, biochemical, electrical, mechanical, and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Description of Embodiments

Wearable technology is booming in neuroscience research. Mobile devices, apps, and body-worn sensors have the ability to monitor gait, body temperature, sleep-wake cycles, heart rate, and more. Data from these devices can be combined with contextual data, behaviors, and interventions in real time. While this technology is increasing in the world around us, it is lacking in rodent research. There is currently no option to determine activity, heart rate, sleep, glucose, caloric intake, blood pressure, or any other physiologic change within one device. Because of this, researchers are forced to choose one device and one data point to measure or focus on for the duration of the study.

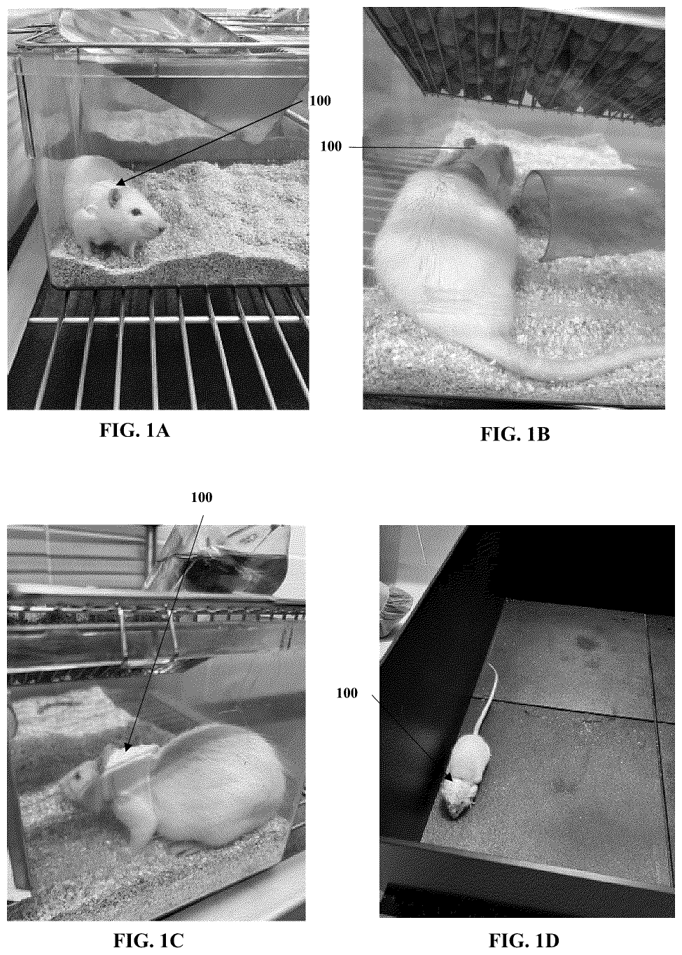

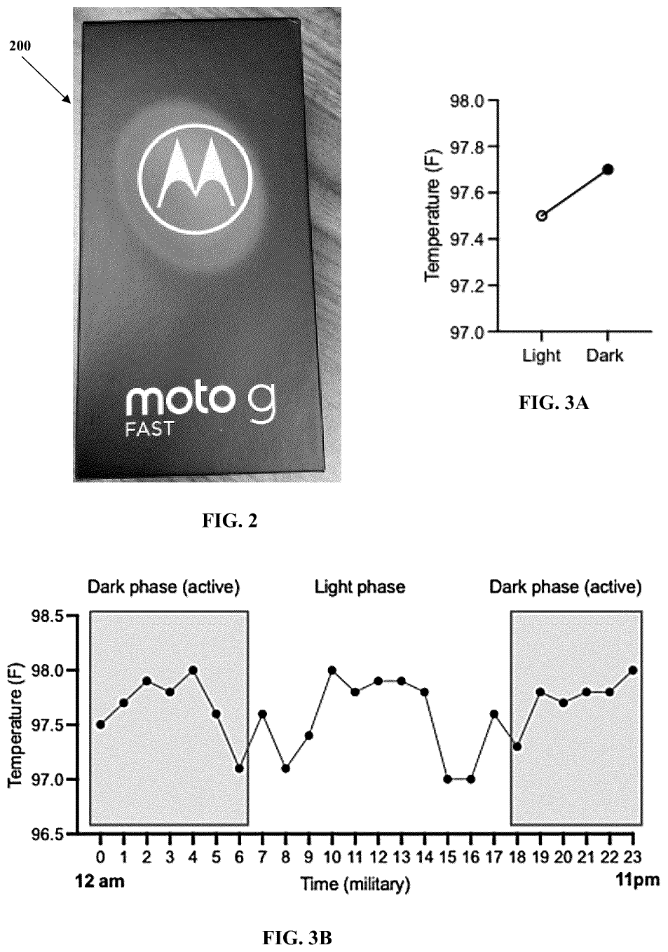

Generally speaking, the wearable tracker 100 comprises an electronic device that can be worn around the neck and/or torso of a subject, as shown in FIGS. 1A-1D. The subject may be an animal, rodent, monkey, ape, pig, or human. The wearable tracker comprises a microprocessor and a wireless connection to track health data across the lifespan of the animal. The wearable tracker measures heart rate, body temperature, respiration, glucose, blood pressure, caloric intake, monitor sleep, movement and social interactions through a combination of sensors.

The wearable tracker changes how aging studies are performed in rodents and creates better preclinical studies for scientists. The wearable tracker collects continuous data in a natural, enriched, or stressful environment. The wearable tracker automates recording of rodents through home-cage monitoring, which allows researchers to collect continuous data without disturbing the animal. The wearable tracker covers all measures into one sensor device, records physiologic changes correlated to brain changes, and is non-invasive. This technology is more efficient, longitudinal and potentially cost-effective as the researcher will not have to buy multiple devices.

In one embodiment, the sensor for rodents is between about 5 grams and 10 grams, or between about 6 grams and 9 grams, or about 7 grams. The wearable tracker records data in rodents and mice to monitor aging, metabolic/nutrition, brain injury, chronic illness research, and the like. The wearable tracker can uncover disease mechanisms by being able to track health data across the mouse lifespan while it is experiencing stress, isolation, or disease, could help to create novel therapeutics. In one embodiment, the wearable tracker could also help mouse colonies with normal colony maintenance as over 120 million mice and rats are used annually in research.

In one embodiment, the wearable tracker includes sensors in a housing and the housing includes an attachment body to attach the housing to the rodent. In one embodiment, the attachment body is Velcro, a zipper, a button, or a hook and loop mechanism. The wearable tracker sends sensor data to a cell phone, which may be set up in the rodent room to collect continuous body temperature data. In one embodiment, the rodents habituate for a week with the wearable tracker and then the rodents are placed in a behavior tests to look at changes in different environments.

The size of the wearable tracker may be reduced in half or more. The wearable tracker will include a plurality of data measurements. The wearable tracker includes a long lasting battery life. However, all sizes may be made as to be able to test on pigs and monkeys as these species are also very important for neuroscience research. In one embodiment, the wearable trackers may be used in humans as a health tracker for aging/chronic disease research. The wearable tracker can also correlate with brain measures and sociability.

Sensors to be used can include, among others: Accelerometer (movement and activity), temperature, heart rate, heart rate variability, O₂ consumption, electrochemical (glucose and menstrual cycle), respiration (sleep), sociability (how close to another rodent).

In order for these sensors to be used with the rodents, for example as compared to how previously done with human devices, they will be smaller and used to measure the same processes as in humans (so as to be translational). The added advantage is to manipulate rodents more easily than humans. It will greatly enhance overall longitudinal data collection and allow researchers to record from the periphery and correlate these findings with brain activity.

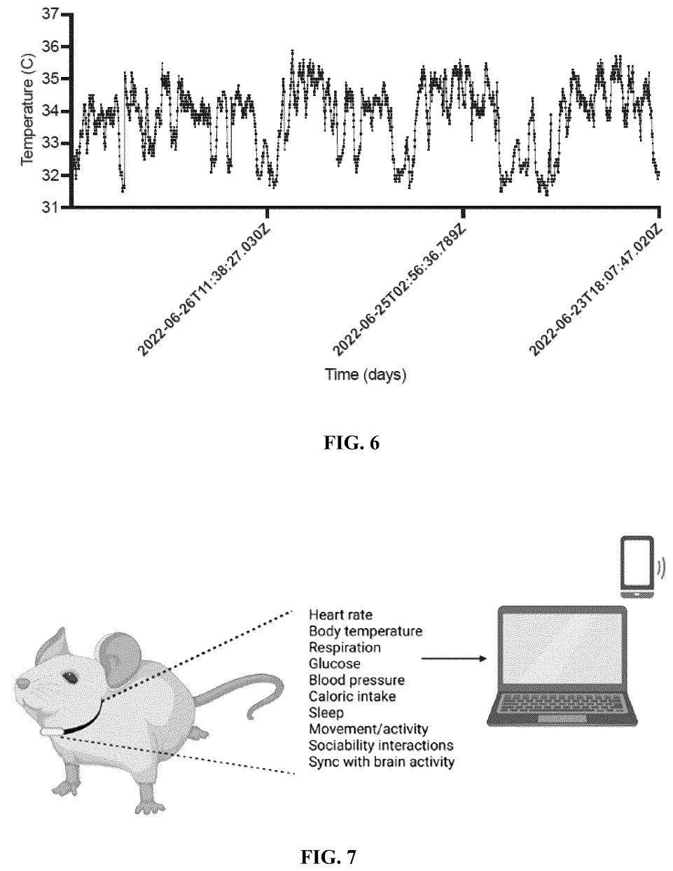

Currently, a scientist has to focus on one measure as tracking rodents with more than one apparatus at a time is costly and inefficient. With the wearable tracker, scientists will be able to examine how the rodent body responds to a drug/therapy or environmental manipulation all in real time. For the interface for data processing and analysis, cloud software is utilized to send data to an application. In one embodiment, a cell phone, tablet, or a basic computer may access the data. FIG. 7 shows the wearable tracker in wireless communication with a computer or smart phone.

In one embodiment, the wearable tracker tracks physiological changes across the lifespan of the mouse for aging and AD research. The wearable tracker tracks Sleep, Heart rate, Body temp, Movement/steps/activity level, Sociability (approaching another mouse), Caloric intake, Cholesterol, Blood pressure, Glucose monitor, Respiration. The wearable tracking syncs with brain measures—neuronal activity. The wearable tracker is operable with software and algorithms to analyze data. A hub or server to collect data wirelessly or wired to a USB. The wearable tracker is non-invasive, collects data longitudinally, collect data while animal is stressed, being given a drug, neutral, social, isolation.

The wearable tracker may be adapted as wearable tech for humans, including but not limited to a smart ring, a smart watch, a fitness tracker, smart glasses, clothing and jackets, smart swim wear, earphones, smart helmets, and other biosensors. Wearable tech is an electronic device that can be worn as accessories embedded in clothing, implanted in skin, or tattooed on skin. Modern wearable tech is defined as incorporating a microprocessor and internet connection. The wearable tracker may be adapted for any animal, including dogs, cats, pigs, and the like as a collar or wearable jacket.

In one embodiment, the wearable tracker sensors monitor gait, body temperature, sleep-wake cycles, vocal quality, eye gaze, heart rate, blood oxygen levels, respiration rate, sweat gland activation, and the like. Data from wearable devices can be combined with contextual data ranging from location and weather tracking to social media usage to ecological momentary assessments, which sample behaviors and experiences in real time. In human neuroscience, wearable versions of laboratory-based neuroimaging modalities, such as wearable electroencephalography (EEG) and functional near-infrared spectroscopy (fNIRS), are prompting ambulatory study designs that more closely mimic everyday experiences. Neuromodulation devices, including implantables, are also changing the landscape of ambulatory perturbation studies; however, alternative embodiments will focus on the highly accessible, non-invasive measures available from easily body-worn devices.

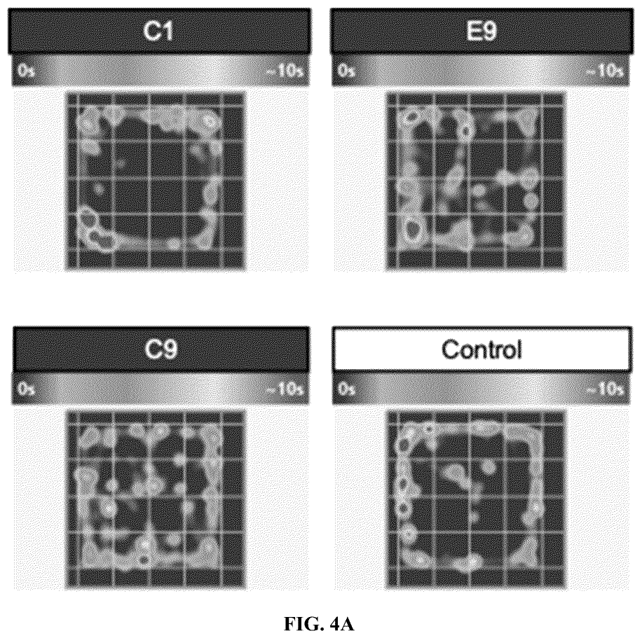

The wearable tracker confirmed changes in temperature depending on time of day. As shown in FIGS. 3A-3B are graphs showing the wearable tracker detecting temperature across 24 hrs; FIG. 3A is a graph showing the average temperature readings while in a home cage during light phase (sleep) and dark phases (wake); and FIG. 3B is a graph showing the continuous temperature reading throughout the day.

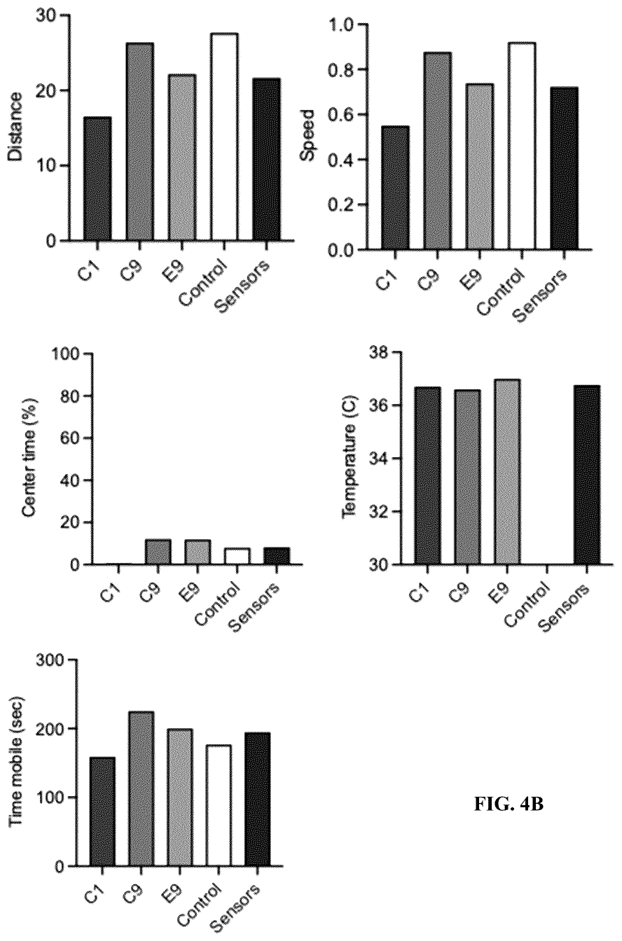

The rodents were placed in an open field and elevated plus maze to determine whether the wearable tracker would impede movement or avoidance behaviors. FIGS. 4A-4B are graphs showing the wearable tracker allowing typical mobility in an open field test; FIG. 4A are representative heat maps of movement comparing rats wearing sensors vs. a control rat without a sensor; and FIG. 4B are distance, traveled, time mobile, and temperature were similar among the groups. No significant differences in distance traveled, temperature, or overall movement patterns in either paradigm were observed.

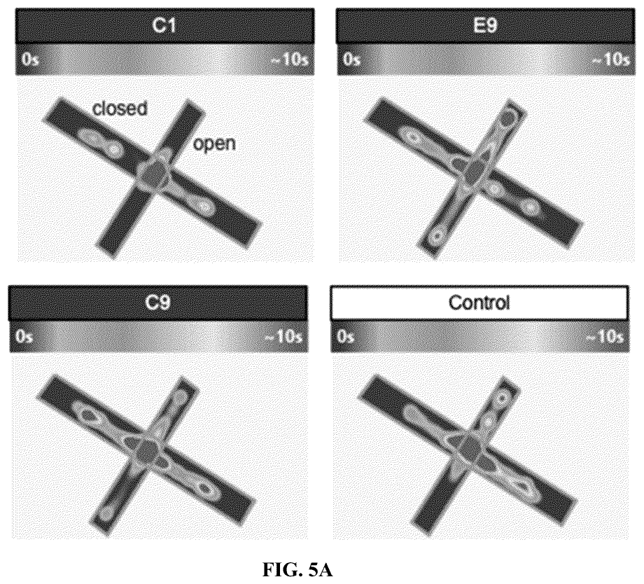

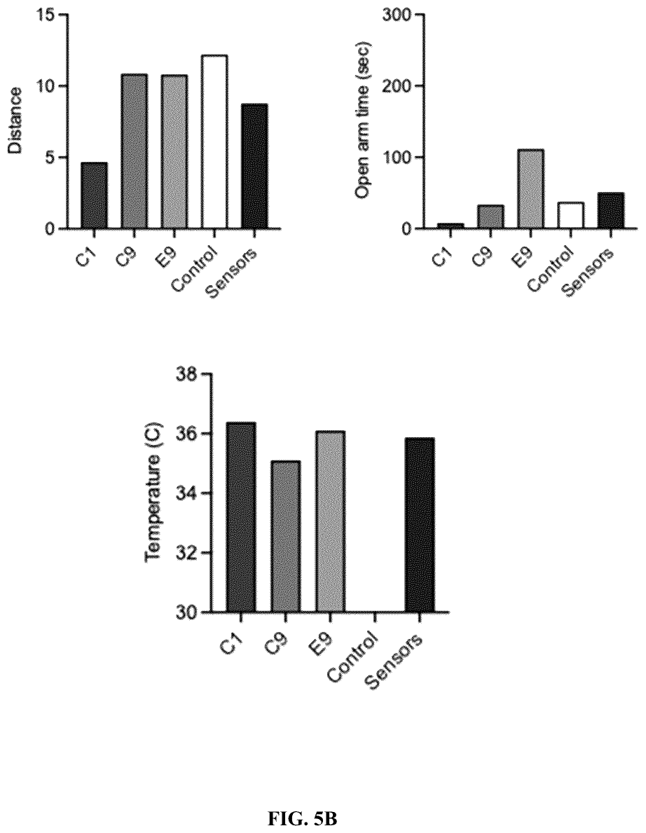

FIG. 5A-5B are graphs showing the wearable tracker sensors allow typical mobility in an elevated plus maze test; FIG. 5A are representative heat maps of movement comparing rats wearing sensors vs. a control rat without a sensor; and FIG. 5B are graphs showing distance, open arm time, and temperature were similar among the groups. FIG. 6 is a graph showing the wearable tracker detecting temperature across multiple days.

The wearable tracker comprises a housing dimension small enough to be worn by a mouse without interfering with their normal behaviors. In one embodiment, the housing dimension is between about 20 mm×about 5.5 mm. The wearable tracker comprises a housing weight light enough to be worn by a mouse without interfering with their normal behaviors. In one embodiment, the housing weight is about 3 g (˜10% of average mouse body weight).

The wearable tracker collects biometrics data continuously. The wearable tracker wirelessly transmits the recorded metrics to a remote processing device. The wearable tracker stores biometrics data while the wireless connection is not available, for later wirelessly transmits the stored biometrics data. The wearable tracker synchronizes captured data with other streams of data captured using other devices, such as brainwave activity. The wearable tracker collects biometrics data: Body temp, Ambulatory Heart rate, Heart rate variability, Respiration, Sleep, Activity.

Wearable Tracker considerations

Mouse physical attributes: Adult mice are typically around 60 mm to 80 mm long (not including the tail) and weigh around 28 g. A target device size of 20 mm×5.5 mm, with a weight of no more than 3 g (11% of body weight) would be ideal. The sensor may be affixed to the mouse using either a custom sleeve, similar to the rat jacket, or using biocompatible adhesive.

The mice are primarily kept in cages with other mice. Periodically, they are removed from these cages and placed in the challenge mazes where much of the research is performed. The research can take place in rooms other than those rooms containing the cages. The sensors should: 1) work regardless of their ability to connect to any hub or other device through which they might automatically download data, 2) should be able to store data for later download. The minimum amount of data they should store is 24 hrs worth of all available metrics. 3) The sensors should be robust enough to withstand potential gnawing (from the mouse wearing the device or others) and 4) the sensors should be waterproof and dustproof to withstand dousing with water (such as from the drink bottles, or fur cleaning, and the general dust in the environment, such as from bedding, food, shed skin cells etc.).

In one embodiment, the sensor is an ultra-low power device, capable of monitoring continuous temperature and transmitting this data through a gateway device to a cloud-based back-end for storage and further processing. The sensor can operate for up to 12 months, with an additional one-year shelf life, from a single coin cell battery.

### Alternative Embodiments

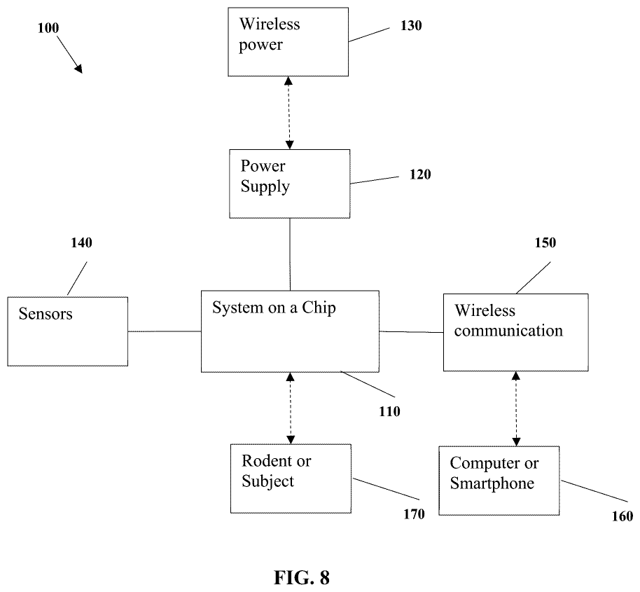

As shown in FIG. 8, the wearable tracker 100 comprises a system on a chip 110, a plurality of sensors 140, a power supply 120, a wireless communication 150, and a computer or smartphone 160. The wearable tracker is housed on a rodent or subject 170. The plurality of sensors measure activity, as well as resting heart rate and heart rate variability, from which sleep and respiration may be derived. These capabilities are enabled via software updates which are stored on the system on the chip 110 and validated through testing and examples indicated below. In one embodiment, a temperature sensor and a system on a chip (SOC) includes storage to store biometric data. In one embodiment, the size of the power supply is reduced such that the wearable tracker can be effectively worn by the rodent or subject. The system on the chip 110 includes a circuit board and electronics that are very small already, residing on a circuit board that is approximately 20 mm long by 10 mm wide, according to one embodiment.

### Wireless Communication:

In one embodiment, the wireless communication is Bluetooth 5.2 for transmission of data from the wearable tracker to the gateway device. Bluetooth 5.2 is the latest Bluetooth protocol. In one embodiment, the wireless communication is on the system on the chip (SOC). It provides far greater range in low-power mode than previous versions of Bluetooth. For one embodiment, the system on the chip (SOC) is used and is operably coupled with firmware for the new power supply and additional sensors.

Alternative embodiments include other wireless communications including Wi-Fi, cellular communications, low-power wide-area networks (LPWAN), or wireless sensor networks. Wi-Fi is a wireless local area network that enables portable computing devices to connect easily with other devices, peripherals, and the Internet. Standardized as IEEE 802.11 a, b, g, n, ac, ax, Wi-Fi has link speeds similar to older standards of wired Ethernet. Wi-Fi has become the de facto standard. Cellular data service offers coverage within a range of 10-15 miles from the nearest cell site. Speeds have increased as technologies have evolved, from earlier technologies such as GSM, CDMA and GPRS, through 3G, to 4G networks such as W-CDMA, EDGE, CDMA2000, or 5G. Low-power wide-area networks (LPWAN) bridge the gap between Wi-Fi and Cellular for low-bitrate Internet of things (IoT) applications. Mobile-satellite communications may be used where other wireless connections are unavailable, such as in remote locations. Wireless sensor networks are responsible for sensing noise, interference, and activity in data collection networks, which allows the detection of relevant quantities, monitor and collect data, formulate clear user displays, and to perform decision-making function.

### Power Supply

In one embodiment, the power supply includes a power harvesting antenna tuned to a particular radio frequency (from about 1.8 to about 4.6 GHz range) to perform near-distance, non-contact, wireless charging of the device. In other embodiments, the power harvesting antenna includes higher frequencies pass more energy and reduce transmission effectiveness. The power harvesting antenna includes two physical components, a plugged-in power emitter and a miniaturized power receiver on the wearable tracker. The power harvesting antenna achieves wireless power up to about 1 ft. In other embodiments, the power harvesting antenna is a WattUp® EN2223 wireless power system.

In one embodiment, the power emitter is placed outside the rodent cage near the water bottle to minimize the distance for recharging, because power dissipates at 1/distance squared. The single emitter will be able to concurrently power multiple rodent devices within the cage.

The power receiver will incorporate a small antenna and solid-state capacitor to store the power. The antenna coil is approximately the size of a dime. Projected time to recharge is less than 6 minutes over a 24-hour time period. This is achieved by combining the over the air power with the extremely low power consumption of the sensor. The sensor firmware will run in a ‘sleep’ state to collect biometrics and only increase power for approximately 5 milliseconds when the sensor is ready to transmit a Bluetooth beacon package, approximately once every 5 minutes.

Wireless power transfer (WPT), wireless power transmission, wireless energy transmission (WET), or electromagnetic power transfer is the transmission of electrical energy without wires as a physical link. In a wireless power transmission system, a transmitter device, driven by electric power from a power source, generates a time-varying electromagnetic field, which transmits power across space to a receiver device, which extracts power from the field and supplies it to an electrical load. The technology of wireless power transmission eliminate the use of the wires and batteries, thus increasing the mobility, convenience, and safety of the wearable tracker. Wireless power transfer is useful to power electrical devices where interconnecting wires are inconvenient, hazardous, or are not possible.

Wireless power techniques mainly fall into two categories, near field and far-field. In near field or non-radiative techniques, power is transferred over short distances by magnetic fields using inductive coupling between coils of wire, or by electric fields using capacitive coupling between metal electrodes. Inductive coupling is the most widely used wireless technology; its applications include charging handheld devices like phones and electric toothbrushes, RFID tags, induction cooking, and wirelessly charging or continuous wireless power transfer in implantable medical devices like artificial cardiac pacemakers, or electric vehicles.

In far-field or radiative techniques, also called power beaming, power is transferred by beams of electromagnetic radiation, like microwaves or laser beams. These techniques can transport energy longer distances but must be aimed at the receiver.

### Sensors:

The plurality of sensors includes temperature sensors, a 3-axis accelerometer, an ambulatory heart rate (HR) data collector. In one embodiment, the accelerometer data is collected by a Bosch 3-axis accelerometer. To collect HR based data, ECG signals are collected by coupling a stainless-steel temperature probe to an amplifier, which then extracts heart rate information, according to one embodiment. As an alternative embodiment to collect heart rate information, if the ECG readings do not pass further feasibility testing, light-based modalites gather a PPG signal directly for the wearable tracker.

A temperature sensor is a device that detects and measures hotness and coolness and converts it into an electrical signal. A broad range of temperature sensors may be used including thermistors, Resistance Temperature Detector (RTD), Thermopile Infrared Temperature Sensors, thermocouples, digital temperatures, Negative Temperature Coefficient (NTC), and thermopiles. RTD sensors are used to measure temperature by changing resistance proportional to the temperature. Thermopile sensors are designed to measure temperature from a distance by detecting an object's infrared (IR) energy. TE Connectivity (TE) is a leading designer and manufacturer of high precision discrete NTC thermistors, probes, and assemblies. An NTC thermistor is a temperature sensor that uses the resistance properties of ceramic/metal composites to measure the temperature. NTC sensors offer many advantages in temperature sensing including miniature size, excellent long-term stability, high accuracy and precision. Digital temperature sensors provide 0.1° C. accuracy. The Temperature System Sensors (TSYS) are miniature packages designed specifically for tight spaces, and respond quickly to changes in process temperature. An optimized microcircuit design allows fast conversion times along with very low power consumption.

Information from Sensors:

From the plurality of sensors, additional metrics are determined, including sleep information and heart rate variability (HRV). Sleep information is gathered by analyzing the accelerometer data. For every 5-minute period, the percentage of time spent across 10 bins of movement is analyzed, which provides low levels of movement data, including the chest expansion from breathing, to ensure that the wearable tracker is being worn, as well as high levels of activity that would mark physical movement throughout a cage. The signature of mostly low-level movement infers sleep. HRV will be analyzed by looking at the variability between heart beats over the course of a minute during times when the animal is sleeping. The accelerometer determines the period of sleep, which then triggers the heart rate sensor at a high sampling rate to measure the precise periods between heartbeats.

### Mechanical Design:

In one embodiment, the mechanical design for the wearable tracker comprises a long, thin housing that will cross the heart of the mouse, being worn on their chest or back. The wearable tracker includes a housing dimension of 20 mm long and 5.5 mm wide, with a weight target of approximately 3 g, as shown Table 1.

TABLE 1
Summary of the main components

| Component | Size | Weight | |
|---|---|---|---|
| Nordic 52832 SOC | 3.0 mm × 3.2 mm | 0.287 | g |
| Bosch Accelerometer | 2 mm × 2 mm × 0.65 mm³ | 0.23 | g |
| Digital Temperature | 1.5 mm × 1 mm | not available | |
| Sensor | | | |
| WattUp ® | 1.69 mm × 1.39 mm × 0.5 mm³ | not available | |
| wireless power | | | |
| receiver | | | |
| Solid state | 5.3 mm × 5.3 mm × 6 mm | 0.18 | g |
| capacitor | | | |
| Housing | ~5.5 mm × ~20 mm | ~0.43 | g |

The reduction in size over the current sensor will be achieved by reducing the housing weight and size, removing the 32 mm diameter battery, and developing a lighter weight circuit board. The current design without housing and battery weighs ˜1.5 g, according to one embodiment.

FIG. 7 includes the properties of the wearable tracker: Non-invasive, Collect data continuously and longitudinally, Cost-efficient, Small/wearable.

### Modules and Algorithms

The wearable tracker includes computer-readable instructions operable to run modules or algorithms to score & predict epileptic events. The wearable tracker collect data from male and female mice, and records types of epilepsy (genetic, induced, mild TBI) (10 mice within each group). The wearable tracker will track mice across their lifespan.

Additional input data into the algorithm is developed by scanning brain tissue for other biomarkers. Algorithms are based on data collection to quantify different types of epileptic events as well as measure ambulatory heart rate and heart rate variability, respiration, sleep, and activity.

The algorithm(s) based on collected data to predict epileptic events.

The algorithms are validated against commonly used models of behavioral and EEG assessments for epilepsy, according to one embodiment.

The wearable tracker accurately scores and predicts seizure activity across the lifespan of a subject. The algorithms are validated against commonly used models of behavioral and EEG assessments for epilepsy. Predictive markers, longitudinal characterization of Post Traumatic Epilepsy, development of novel technology to improve PTE research with the potential to affect many classes of epileptic patients both military and non-military.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Algorithms to Score & Predict Epileptic Events

In Example 1, the goal is to use the wearable tracker to collect data from male and female mice with and without genetic epilepsy to determine baseline measures in behavior and physiology. Algorithms will be developed based on data collection to quantify different types of epileptic events as well as measure ambulatory HR and HRV, respiration, sleep, and activity.

Lastly, an algorithm to predict epileptic events is developed. Sex as a biological variable: Sex differences are evident in epilepsy with men exhibiting greater overall seizure susceptibility than women, while women exhibit greater fluctuations in seizure susceptibility (12). Therefore, male and female mice will be compared.

Overall experiment: All mice will go through an initial baseline behavior testing outlined below at 2 months of age. Then there will be two arms for this study: Arm 1) Scn1a (genetic epilepsy) mice will be given a sham or TBI surgery to assess spontaneous seizures before and after TBI (post traumatic epilepsy; PTE model). 24-hour video recording will occur in the home cage. This recording will last 1 week. Mice will then be sacrificed, and brain tissue taken for analysis of atrophy and inflammatory markers. Arm 2) Control mice will undergo sham or TBI surgery. After recovery, mice will either be injected with saline or kainic acid (KA chemically induced seizure; PTE). Seizure onset and severity will be recorded in an open field. Hand scoring of the seizure severity will also be completed. KA injections will be given every other day for 1 week. Mice will then be sacrificed and brain tissue taken for analysis of atrophy and inflammatory markers.

Mice: Male and female 129s6 (Control), Scn1a (genetic epilepsy model). Groups: control-sham-saline, control-sham-KA, control-TBI-saline, control-TBI-KA, Scn1a-sham, Scn1a-TBI. ** Control saline mice will also be used as a control comparison against Scn1a group to reduce mouse groups.

Baseline Behavior design: Biosensors will be placed on all mice at 2 months of age. Mice will habituate to the sensors in their home cages for 1 week. First, physiological measurements in their home cages will be recorded. Next, anxiety-like behavior will be assayed while recording measurements using: 1) Elevated Plus Maze (EPM): Mice will be individually placed in the center of the maze facing an open arm and allowed to explore the maze for 5 min. The duration of time spent in the open arms versus the closed arms and time in center will be used as anxiety indices, 2) Marble Burying: Mice will be placed in a square cage with sawdust bedding. Sixteen marbles are set in a descending horizontal pattern on top of the bedding. After 20 minutes, mice are removed, and the marbles buried are counted. Because anxiolytics such as diazepam are shown to decrease burying, the number of marbles buried will be used as a measure of anxiety, 3) Novelty Suppressed Feeding (NSF): Mice will be food restricted for 24 h and placed in a brightly lit arena with a food pellet placed in the center. Latency of the animals to begin eating the pellet will be recorded and used as a measure of anxiety, 4) Light Dark Box: Mice will be placed on the light side of a Light Dark Box chamber, the middle partition is open and mice will be free to roam the light and dark side of the chamber for 10 minutes, and 5) Open Field (OF): Mice will be placed into an OF box in a dimly lit room for 10 min. Time spent in the center (middle 50% of the arena) will be measured as an indicator of anxiety-like behavior. All anxiety-like tests will be recorded using Ethovision software.

Working, short- and long-term memory will be assayed while recording measurements using: 1) Barnes Maze (BM): The BM is a circular table with 20 open holes and 1 escape box. On day 1 mice will be habituated to the maze and allowed to explore for 5 minutes. Mice will be placed in the escape box at the end of the trial. Acquisition training will occur on days 1-10. After habituation, mice will be placed in the center of the maze and given 3 minutes to learn where the escape box is located. They will complete 2 trials per day. If the mouse fails to find the escape box, it is placed in the box by the researcher. 24 hrs after the final acquisition day, mice undergo a 1-minute probe trial in which the escape box is removed. For the probe trial, latency and distance traveled to the target hole are measured. 2) Novel Object Recognition (NOR): The paradigm will consist of five 5-min exposures with 3-min intertrial intervals. Exposures 1-4 will be habituation sessions with 2 objects placed symmetrically on either end of the arena about 5 cm away from the wall. During exposure 5, one of the objects will be replaced with a novel object, and 3) Contextual Fear Conditioning (CFC): Mice will be administered 3-shock CFC on training day. Five days later, mice will be re-exposed to the training context without shock and freezing behavior will be measured.

Measuring seizures experimental timeline: After baseline data collection, the wearable tracker measures physiologic changes before, during, and after epileptic events (genetic, induced, after TBI). At 4 months of age Cohort 1 (Scn1a) mice will either be given a sham surgery or TBI. Spontaneous seizures will be measured in the home cage. At 4 months of age Cohort 2 (Control) mice will either be given a sham or TBI surgery followed by saline or KA to induce seizures. Seizure severity will be measured in the open field.

Inducing seizures: chemically induce seizures by using subcutaneous (s.c.) injection of kainic acid (KA) (12 mg/kg) as has been previously achieved by Dr. Hunsberger (13,14). Control mice will be given saline. Mice will be placed in an open field arena and seizure severity will be graded by blinded observers in 5-minute increments using the 6-step scale (13,15,16). Seizure scores: 1, mouth and facial movement; 2, head nodding; 3, forelimb clonus; 4, rearing with forelimb clonus; 5, rearing and falling with forelimb clonus; 6, tonic-clonic seizures; 7, mortality.

Genetic seizure model: Mice homozygous for the Scn1a knockout allele exhibit seizures and die by postnatal day 16. Therefore, heterozygous Scn1a knockout mice will be used on a 129s6 background strain (Jackson Laboratory; 12S-ScnlatmlKea/Mmjax) to detect mild spontaneous seizure (17). Mutations in Scn1a, a gene that encodes the alpha subunit of voltage-gated sodium channel Nav1.1, are associated with several epilepsy syndromes such as Dravet syndrome and epileptic encephalopathy in humans (18). This model was chosen due to its translational impact as Scn1a is one of the most common and well-researched epilepsy genes (3).

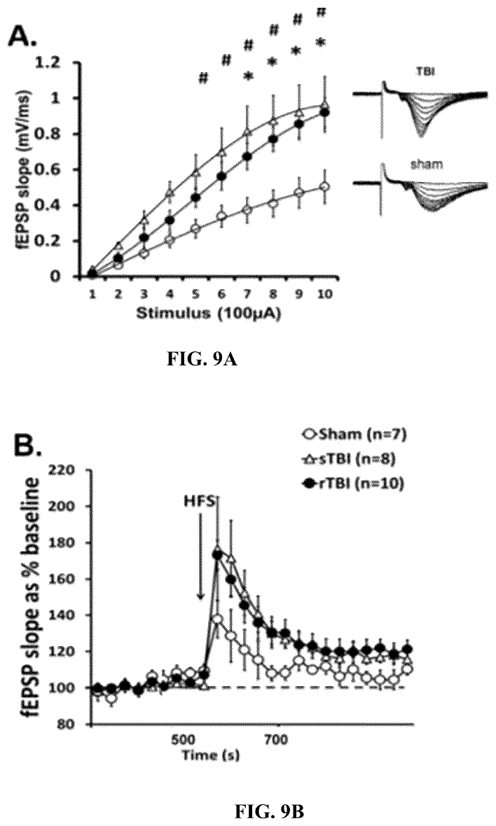

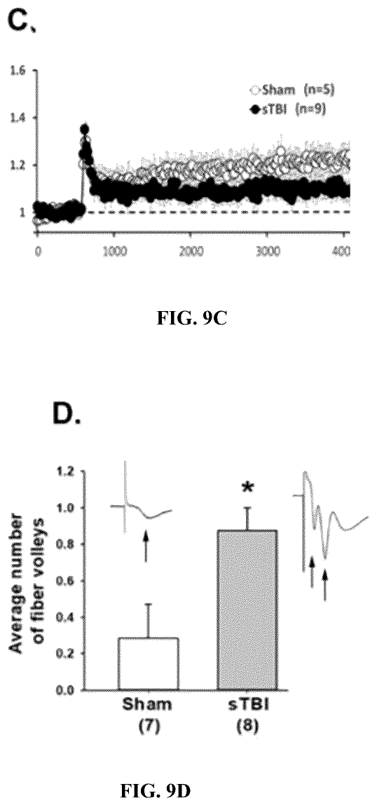

Traumatic brain injury: a cortical controlled impact (CCI) device will be used to deliver repeat closed-head impacts to mice. This model best translates to a clinical situation in which the majority of people suffer multiple concussive events and not one severe trauma. In collaboration with the Stutzmann Lab, accurately perform CCI TBI, as single and repetitive TBI results in synaptic changes throughout the hippocampus, specifically hyperexcitability within the CA3 which could contribute to seizure susceptibility (FIGS. 9A-9D) (19,20). Mice will receive TBI using a controlled cortical impact device (Leica). A representative brain tissue image is shown in FIG. 10. TBI is delivered every other day for a total of five impacts. For every impact and SHAM procedure, mice are anesthetized in 2% isoflurane and rest on a foam pad, head not fixed. The 5 mm impactor tip is placed right of midline and centered between the caudal edge of the eye, and the rostral edge of the ear, approximately 2 mm posterior and 1.5 mm lateral to Bregma. This places the piston over the right sensorimotor cortex without opening the skin. The piston is lowered until contact is made with the top of the head, then primed for impact using the “retract” feature. The device then impacts the surface of the head at a depth of 2 mm, velocity of 5 m/sec, and with a 300 ms dwell time. For the SHAM procedure, the piston is aligned identically to the injury procedure, but then moved away from the head of the animal before activation, resulting in the same amount of time spent on the foam pad under anesthesia, and the same noise from triggering the CCI device, sans impact. After the piston is triggered, mice are immediately removed from anesthesia, weighed, and placed on their back to assess righting reflex before returning to their home cage.

### Develop Algorithms to Predict Seizure Onset:

The algorithms combine data from the sensors gathered on an electronic platform, a plurality of sensors, as well as observed data in a central data warehouse. All data will be timestamped to align various measures. Observational data will be annotated with the observed epilepsy events, sleep, activity, duration and level of anxiety, and memory. Features from the sensor data will be extracted and pre-processed for baseline correction, artifact removal, and signal normalization. The next step will be algorithm training for extracting direct physiological biometrics, such as heart rate, heart rate variability, respiration and movement. The plurality of sensors, such as the Ethovision system in training accelerometer data to develop baseline ‘normal’ metrics for heart rate, heart rate variability, respiration, and movement. The analysis of the data will determine not only the measure of the data, such as heart rates per minute, but also the conditions to achieve the measurement, such as defining times with low movement. The ‘normal’ movement, heart rate, and respiration baseline will allow to filter normal biometrics from epilepsy events in the accelerometer. Algorithm training for a predicting an epilepsy event will utilize machine learning techniques, such as reoccurring neural networks to capture the time dimension of the data. While the data for training and validation sets is split to get an idea of the model fit, the final training will be run on the entire data set, as prospective validation will occur in Example 2. Analysis will be run on each arm of the study independently as well as combined to understand overlap between the two arms.

### Hypothesis:

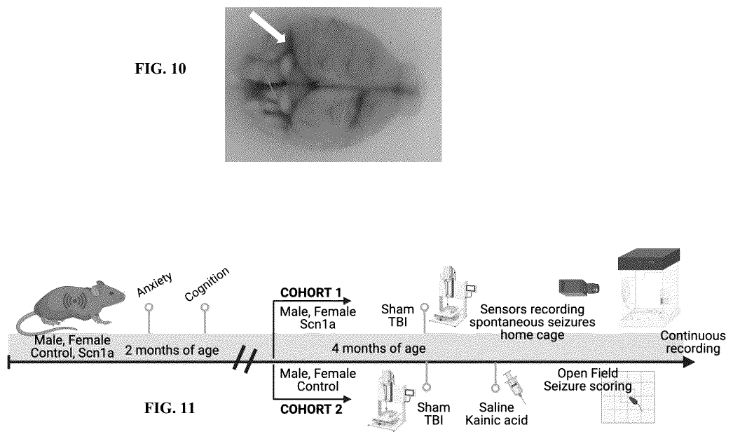

With this experimental behavioral design, as shown in FIG. 11, the proof of concept for the wearable rodent health tracker is provided. The physiological differences between control, Scn1a, KA- and TBI-induced, male, and female mice will be identified. Recently, preliminary data showed changes in temperature while the animal is the dark vs light phase and further prove that rodents move freely in behavior tests with these sensors attached. Therefore, 1) The wearable sensor will not impede movement or affect behavior; 2) The wearable sensor will accurately detect physiological changes while the animal is performing behavior tasks; 3) there will be differences in physiologic measures between control and epileptic mouse models and that TBI will further exacerbate seizure susceptibility; 4) mice will exhibit sex-specific physiological measurements.

### Potential Pitfalls and Alternatives:

Although the wearable tracker will not impede movement or behavior, the mouse may chew or scratch at the device. If this occurs, the wearable tracker may include a medical adhesive without a strap. Although issues with the algorithms are not expected if measurement one of the sensor variables is inaccurate, the electronic device will still be functional. A strong enough correlation with control sensors, either because of lack of data or sensor performance. A variety of behavioral tests will be used to ensure the sensor can be used in many different environments. Therefore, if the wearable tracker impedes a test, alternative tests will be used. A specific algorithm changes for sex or age will not be needed, but this may be required, and if so, would necessitate additional animals. Because genetic rodent models of epilepsy are at risk for early mortality, different models to measure different age groups may be used. For example, the B6.129 (Cg)-Cntnap2^{tm1Pele}/J knockout mice exhibit hyperactivity and spontaneous seizures or the 129S-Kcnab2^{tm1Kme}/NystJ is a novel potassium channel knockout mouse that has yet to be characterized and could exhibit milder seizure phenotypes (Jax labs strain 017482; Jax labs strain 037894). A second option is to test the genetic models at 2 months with TBI. Lastly, the J20 mouse model of Alzheimer's disease (hAPP-J20/Tau−/−) (21) may be used, which exhibit brain hyperexcitability and sporadic seizures.

Summary: Completion of Example 2 will result in wearable health tracker data and algorithms that can then be used against industry standard devices.

### Example 2: Validate Algorithms Against Commonly Used Models of Behavioral and EEG Assessments for Epilepsy

In Example 2, the goal is to validate this technology and algorithms against common techniques already available on the market.

Confirmation of physiologic measurements: Validation in mice will be completed using the measurements from the animal in comparison to the measurements on a known sensor. Specifically, to confirm temperature, a rectal probe thermometer will be used. To confirm HR and HRV, a rodent ECG sensor will be used. For movement measurements, Ethovision software and OF paradigm will be used to compare the distance moved and time mobile. The Piezo sleep boxes will be used to compare sleep statistics with the sensor measures. Lastly, existing implantable EEG and telemetry devices will be used on the market to compare the metrics.

Ethovision (Noldus) is a behavioral tracking system to monitor distance traveled, velocity, movement, and many other behaviors. A video camera is mounted above the testing apparatus and is attached to a computer with dedicated Ethovision software which then tracks and records the animal. Movement data from Ethovision will be compared to the sensor data to validate movement algorithms.

Piezo sleep boxes (Signal Solutions) measure sleep and activity in aged and Alzheimer's disease animals (22). These boxes use sensors on the floor of the cage to track movement, respiration, and temperature to detect sleep versus wake bouts. Sleep data from Piezo will be compared to the sensor data to validate sleep algorithms.

Telemetry devices: a common telemetry system from Data Science Instruments (DSI) will be used. A standard implant is the PhysioTel transmitter (TA10ETA-F20) which will measure EEG, ECG, EMG, and physical activity and temperature (23). The receiver RPC-picks up the telemetered data from the implant, the data exchange matrix serving as a multiplexer, and the data acquisition and analysis processing core unit. The Hunsberger lab has wide-ranging experience with surgical techniques including, implantation of osmotic minipumps, lens implants for miniscope imaging, viral injections, ovariotomy, microelectrodes, and optogenetic probes. Surgical implantation with a midline incision of the scalp from forehead will be made and a s.c. pouch will be created along the lateral flank close to the ventral abdominal regions. The transmitter will be fixed at the skin using the transmitter's suture tab and a stich. An EEG electrode will be implanted into the dorsal CAI region while epidural differential electrodes are placed over the motor cortex and cerebellum. The implant will be covered with Metabond cement.

Measuring seizures experimental timeline: All groups and methods of seizure induction will be replicated from Example 1. At 2 months of age mice will be implanted with the telemetry device, biosensors will be attached, and behavior will be tested in all previous paradigms. Seizure will then be induced chemically and/or with TBI.

Effect of benzodiazepines: To test the sensor feasibility in response to pharmacological methods, the same cohort of mice will be injected with saline or Alprazolam. Benzodiazepines are often used for treatment of seizures. With continuous measurements, the physiological effects of drug before, during, and after treatment will be compared.

Potential pitfalls and alternatives: If implantation of the DSI telemetry devices proves difficult (the combination weight of the wearable tracker and the DSI device), additional verification tools may be added. For example, Home cage infrared motion detectors can be used (STARR Life Sciences Corporation), activity cages from Ugo Basile, and TSE metabolic cages which can measure O2 consumption, food intake, physical activity, and running wheel activity. Although unlikely there is a possibility that the biosensors do not match the current market devices. From the validation data, the necessary changes or adjustments in wearable tracker, algorithm, or data collection will be made.

Summary: Completion of Example 2 will result in a wearable health tracker that has been demonstrated as fit for purpose for use in rodent models of epilepsy.

Statistics: All data will be analyzed using JMPC software (SAS Institute, Cary, NC). Data will be analyzed using two-or three-way (injury x injection x sex; group x injury x sex) ANOVA with repeated measures (across days) when appropriate. Tukey post-hoc tests will be performed where appropriate if ANOVA interaction is significant. Alpha will be set to 0.05 for all analyses. The previous studies and a power analysis set at 0.80 an effect size of 0.5 revealed that a sample size of ˜5-10 mice per group is needed to reach significance. All experimenters will be blinded to group when running and scoring behavior and organizing data until statistics are performed.

### Example 3: Protocol for Different Experiments for Alzheimer's Disease

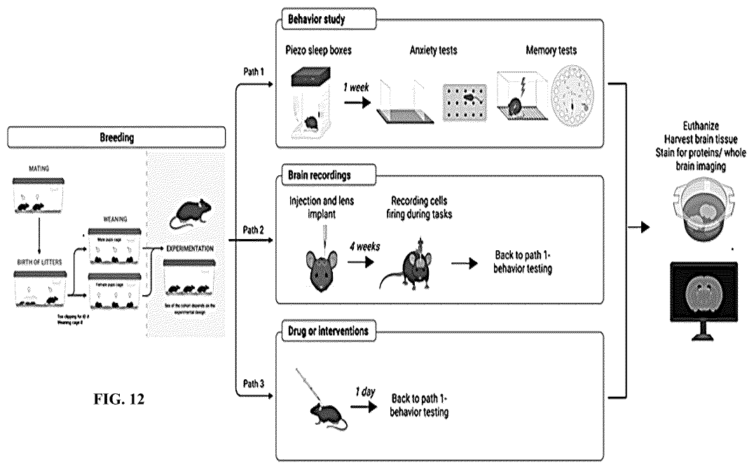

Potential comparisons: for Control vs Alzheimer mice, Ages, Sexes, Mice reared in enriched environment vs poor environment, and Stressed vs. non-stressed. FIG. 12 is a schematic flow diagram of the experimental design for Alzheimer's disease.

Animals ID via toe clips and weaned into separate cage #.

All cages on one rack in one room. For behavior testing-moved to a new room.

Data collected through each recording program and exported to excel file.

Excel file data saved on google drive.

Data input into graphing program and stats program via csv or copy and paste.

Data for calcium imaging-collected through Wi-Fi daq box then stored to an external hard

drive. Data is processed through Inscopix programs and Python in other embodiments.

### Abbreviations

ANN: artificial neural network, BM: barnes maze, CCI: cortical controlled impact, CFC: contextual fear conditioning, Ctrl: control, DSI: data science instruments, ECG: electrocardiogram, EEG: electroencephalogram, EPM: elevated plus maze, g: grams, HR: heart rate, HRV: heart rate variability, i.p.: intraperitoneal, KA: kainic acid, LD: light dark box, LTP: long term potentiation, MB: marble burying, NOR: novel object recognition, NSF: novelty suppressed feeding, OF: open field, PTE: post-traumatic epilepsy, rTBI: repeat traumatic brain injury, s.c.: subcutaneous, Scn1a: Sodium channel protein type 1 subunit alpha (genetic epilepsy mouse model), sTBI: single traumatic brain injury, TBI: traumatic brain injury.

### System

As used in this application, the terms “component” and “system” are intended to refer to a computer-related entity, either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

Generally, program modules include routines, programs, components, data structures, etc., that perform particular tasks or implement particular abstract data types. Moreover, those skilled in the art will appreciate that the inventive methods can be practiced with other computer system configurations, including single-processor or multiprocessor computer systems, minicomputers, mainframe computers, as well as personal computers, hand-held computing devices, microprocessor-based or programmable consumer electronics, and the like, each of which can be operatively coupled to one or more associated devices.

The illustrated aspects of the innovation may also be practiced in distributed computing environments where certain tasks are performed by remote processing devices that are linked through a communications network. In a distributed computing environment, program modules can be located in both local and remote memory storage devices.

A computer typically includes a variety of computer-readable media. Computer-readable media can be any available media that can be accessed by the computer and includes both volatile and nonvolatile media, removable and non-removable media. By way of example, and not limitation, computer-readable media can comprise computer storage media and communication media. Computer storage media includes volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or any other medium which can be used to store the desired information and which can be accessed by the computer.

Communication media typically embodies computer-readable instructions, data structures, program modules or other data in a modulated data signal such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer-readable media.

Software includes applications and algorithms. Software may be implemented in a smart phone, tablet, or personal computer, in the cloud, on a wearable device, or other computing or processing device. Software may include logs, journals, tables, games, recordings, communications, SMS messages, Web sites, charts, interactive tools, social networks, VOIP (Voice Over Internet Protocol), e-mails, and videos.

In some embodiments, some or all of the functions or process(es) described herein and performed by a computer program that is formed from computer readable program code and that is embodied in a computer readable medium. The phrase “computer readable program code” includes any type of computer code, including source code, object code, executable code, firmware, software, etc. The phrase “computer readable medium” includes any type of medium capable of being accessed by a computer, such as read only memory (ROM), random access memory (RAM), a hard disk drive, a compact disc (CD), a digital video disc (DVD), or any other type of memory.

### REFERENCES

1. Carbone L (2021): Estimating mouse and rat use in American laboratories by extrapolation from Animal Welfare Act-regulated species. Sci Rep 11:493.
2. Waterston R H, Lindblad-Toh K, Birney E, Rogers J, Abril J F, Agarwal P, et al. (2002): Initial sequencing and comparative analysis of the mouse genome. Nature 420:520-562.
3. Marshall G F, Gonzalez-Sulser A, Abbott CM (2021): Modelling epilepsy in the mouse: challenges and solutions. Dis Model Mech 14. https://doi.org/10.1242/DMM.047449
4. Golub V M, Reddy D S (2022): Contusion brain damage in mice for modelling of post-traumatic epilepsy with contralateral hippocampus sclerosis: Comprehensive and longitudinal characterization of spontaneous seizures, neuropathology, and neuropsychiatric comorbidities. Exp Neurol 348. https://doi.org/10.1016/J.EXPNEUROL.2021.113946
5. Charles river (2023): Device implants. Retrieved Jun. 18, 2023, from https://www.criver.com/sites/default/files/noindex/catalogs/rms/device-implants-us-pricing.pdf
6. Nih, Od, Oer, Olaw (2011): GUIDE LABORATORY ANIMALS FOR THE CARE AND USE OF Eighth Edition Committee for the Update of the Guide for the Care and Use of Laboratory Animals Institute for Laboratory Animal Research Division on Earth and Life Studies. Retrieved Jun. 18, 2023, from http://www.nap.edu.
7. Angeleri F, Majkowski J, Cacchiò G, Sobieszek A, D'Acunto S, Gesuita R, et al. (1999): Posttraumatic Epilepsy Risk Factors: One-Year Prospective Study After Head Injury. Epilepsia 40:1222-1230.
8. Weiss G H, Feeney D M, Caveness W F, Dillon L D, Kistler J P, Mohr J P, Rish B L (1983): Prognostic factors for the occurrence of posttraumatic epilepsy. Arch Neurol 40:7-10.
9. Annegers J F, Hauser W A, Coan S P, Rocca W A (1998): A population-based study of seizures after traumatic brain injuries. N Engl J Med 338:20-24.
10. Hauser W A, Annegers J F, Kurland LT (1993): Incidence of epilepsy and unprovoked seizures in Rochester, Minnesota: 1935-1984. Epilepsia 34:453-458.
11. Wang X, Zhong J, Lei T, Chen D, Wang H, Zhu L, et al. (2021): An Artificial Neural Network Prediction Model for Posttraumatic Epilepsy: Retrospective Cohort Study. J Med Internet Res 23. https://doi.org/10.2196/25090
12. Reddy D S, Thompson W, Calderara G (2021): Molecular mechanisms of sex differences in epilepsy and seizure susceptibility in chemical, genetic and acquired epileptogenesis. Neurosci Lett 750. https://doi.org/10.1016/J.NEULET.2021.135753
13. Hunsberger H C, Konat G W, Reed M N (2017): Peripheral viral challenge elevates extracellular glutamate in the hippocampus leading to seizure hypersusceptibility. J Neurochem 141:341-346.
14. Hunsberger H C, Wang D, Petrisko T J, Alhowail A, Setti S E, Suppiramaniam V, et al. (2016): Peripherally restricted viral challenge elevates extracellular glutamate and enhances synaptic transmission in the hippocampus. J Neurochem 138:307-316.
15. Racine R J, Burnham W M I, Gartner J G, Levitan D (1973): Rates of motor seizure development in rats subjected to electrical brain stimulation: strain and inter-stimulation interval effects. Electroencephalogr Clin Neurophysiol 35:553-556.
16. Morrison R S, Wenzel H J, Kinoshita Y, Robbins C A, Donehower L A, Schwartzkroin PA (1996): Loss of the p53 tumor suppressor gene protects neurons from kainate-induced cell death. The Journal of Neuroscience 16:1337.
17. Mistry A M, Thompson C H, Miller A R, Vanoye C G, George A L, Kearney J A (2014): Strain- and age-dependent hippocampal neuron sodium currents correlate with epilepsy severity in Dravet syndrome mice. Neurobiol Dis 65:1-11.
18. Miller A R, Hawkins N A, Mccollom C E, Kearney J A (2014): Mapping genetic modifiers of survival in a mouse model of Dravet syndrome. Genes Brain Behav 13:163-172.
19. McDaid J, Briggs C A, Barrington N M, Peterson D A, Kozlowski D A, Stutzmann G E (2021): Sustained Hippocampal Synaptic Pathophysiology Following Single and Repeated Closed-Head Concussive Impacts. Front Cell Neurosci 15. https://doi.org/10.3389/FNCEL.2021.652721
20. Shim S S, Stutzmann G E (2016): Inhibition of Glycogen Synthase Kinase-3: An Emerging Target in the Treatment of Traumatic Brain Injury. J Neurotrauma 33:2065-2076.
21. Palop J J, Chin J, Roberson E D, Wang J, Thwin M T, Bien-Ly N, et al. (2007): Aberrant excitatory neuronal activity and compensatory remodeling of inhibitory hippocampal circuits in mouse models of Alzheimer's disease. Neuron 55:697-711.
22. Topchiy I, Fink A M, Maki K A, Calik M W (2022): Validation of PiezoSleep Scoring Against EEG/EMG Sleep Scoring in Rats. Nat Sci Sleep 14:1877-1886.
23. Lundt A, Wormuth C, Siwek M E, Müller R, Ehninger D, Henseler C, et al. (2016): EEG Radiotelemetry in Small Laboratory Rodents: A Powerful State-of-the Art Approach in Neuropsychiatric, Neurodegenerative, and Epilepsy Research. Neural Plast 2016. https://doi.org/10.1155/2016/8213878
24. Hunsberger H C, Rudy C C, Batten S R, Gerhardt G A, Reed M N (2015): P301L tau expression affects glutamate release and clearance in the hippocampal trisynaptic pathway. J Neurochem 132:169-182.
25. Hunsberger H C, Weitzner D S, Rudy C C, Hickman J E, Libell E M, Speer R R, et al. (2015): Riluzole rescues glutamate alterations, cognitive deficits, and tau pathology associated with P301L tau expression. J Neurochem 135:381-394.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method of tracking biometrics data comprising:
Using a wearable tracking including an electronic device that is worn by a subject;
Tracking biometrics data by the electronic device, wherein the electronic device includes a microprocessor and a wireless connection to track biometrics data across the lifespan of the subject;
measuring heart rate, body temperature, respiration, glucose, blood pressure, caloric intake, monitor sleep, movement and social interactions through a plurality of sensors on the electronic device;
recording data to monitor aging, metabolic/nutrition, brain injury, chronic illness research; and uncover disease mechanisms by being able to track health data across the subject lifespan while it is experiencing stress, isolation, or disease;
performing behavior tests; and sending the biometrics data wirelessly to a cell phone, a computer, or a table device.

2. The method of claim 1, wherein the wearable tracker is configured to be worn on the neck and/or torso of a subject.

3. The method of claim 2, wherein the subject is selected from the group consisting of an animal, rodent, monkey, ape, pig, or human.

4. The method of claim 3, further comprising performing aging studies on rodents and improving preclinical studies by collecting continuous biometrics data in a natural environment.

5. The method of claim 4, further comprising recording physiologic changes correlated to brain changes.

6. The method of claim 5, further comprising using the wearable tracker on humans for tracking aging or chronic disease.

7. The method of claim 6, wherein the plurality of sensors are selected from the group consisting of: Accelerometer, temperature sensor, heart rate sensor, heart rate variability sensor, O₂ consumption sensor, electrochemical sensor for glucose or menstrual cycles, respiration sensor for sleep, sociability sensor.

8. The method of claim 7, further comprising collecting longitudinal data and correlating the biometrics data with brain activity; and examining how the rodent responds to a drug, a therapy, or an environmental manipulation all in real time; and processing the biometrics data analysis by an application.

9. The method of claim 8, further comprising tracking physiological changes in a subject for Alzheimer's Disease:
using the wearable tracker to track biometrics data selected from the group consisting of: Sleep data, Heart rate data, Body temperature data, Movement/steps/activity level data, Sociability data, Caloric intake data, Cholesterol data, Blood pressure data, Glucose monitor data, or Respiration data;
syncing the wearable tracking with brain measurement and neuronal activity data; and
analyzing the biometrics data and the brain measurement and neuronal activity data with a server system over a period of time in response to a drug or a social interaction; and
wherein the wearable tracker is selected from the group consisting of: a smart ring, a smart watch, a fitness tracker, smart glasses, clothing and jackets, smart swim wear, earphones, smart helmets, a biosensor clothing, an implant, or a tattoo; and the wearable tracker includes a microprocessor and an internet connection.

10. The method of claim 9, further comprising monitoring gait, body temperature, sleep-wake cycles, vocal quality, eye gaze, heart rate, blood oxygen levels, respiration rate, sweat gland activation, location and weather data, social media usage data, or ecological momentary data.

11. A method of collecting biometrics data continuously, comprising:
collecting biometrics data including Body temperature, Ambulatory Heart rate, Heart rate variability, Respiration, Sleep, or physical Activity by a plurality of sensors disposed in a wearable tracker;
storing biometrics data while the wireless connection is not available; and
wirelessly transmits the biometrics data to a remote processing device.

12. The method of claim 11, wherein the plurality of sensors are powered by an ultra-low power device; wherein the plurality of sensors are operably coupled to a system on a chip and a wireless communication device; and the wireless communication is Bluetooth for transmission of data from the wearable tracker to the gateway device.

13. The method of claim 12, wherein the power supply includes a power harvesting antenna tuned to a particular radio frequency to perform near-distance, non-contact, wireless charging of the device; wherein the power harvesting antenna includes a plugged-in power emitter and a power receiver; wherein the power receiver includes a small antenna and solid-state capacitor to store the power; wherein the plurality of sensors includes temperature sensors, a 3-axis accelerometer, an ambulatory heart rate (HR) data collector, light-based sensors to gather a PPG signal.

14. The method of claim 13, wherein the plurality of sensors track sleep data by analyzing the accelerometer data and heart rate variability (HRV) data; wherein the accelerometer data includes a signature of low-level movement infers sleep; and analyzing HRV data by looking at the variability between heart beats over the course of a minute during times when the animal is sleeping; determining the period of sleep by the accelerometer data, which then triggers the heart rate sensor at a high sampling rate to measure the precise periods between heartbeats.

15. The method of claim 14, wherein the wearable tracker includes a long, thin housing wearable on the chest or back of a subject.

16. A method of tracking and predicting epileptic events comprising:
Using a wearable tracker to score & predict epileptic events;
Collecting data from the wearable tracker and recording the types of epilepsy; and
Predicting an epileptic event;
scanning brain tissue for biomarkers and quantifying different types of epileptic events based on the collected data;
wherein the collected data is based on measured ambulatory heart rate and heart rate variability, respiration, sleep, and activity.

17. The method of claim 16, further comprising validating the method of tracking and predicting epileptic events against commonly used models of behavioral and EEG assessments for epilepsy; accurately score seizure activity across the lifespan of a subject by the wearable tracker; wherein the collected data includes epilepsy events, sleep, activity, duration and level of anxiety, and memory; extracting and pre-processing the collected data for baseline correction, artifact removal, and signal normalization.

18. The method of claim 17, further comprising predicting an epilepsy event by machine learning methods to capture the time dimension of the data.
